(19) United States
(12) Patent Application Publication
(10) Pub. No.: US 2007/0031522 A1
(43) Pub. Date: Feb. 8, 2007

(54) Enriched aqueous components of emblica officinalis

(76) Inventors: Ratan Chaudhuri, Lincoln Park, NJ (US); Germain Puccetti, Ossining, NY (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/571,588

(22) PCT Filed: Aug. 13, 2004
(86) PCT No.: PCT/EP04/09109

§ 371 Date: Mar. 10, 2006

Related U.S. Application Data
(63) Continuation-in-part of application No. 10/660,742, filed on Sep. 12, 2003.

(51) Int. Cl. A61K 36/185

(52) U.S. Cl. 424/769

(57) ABSTRACT

In an extraction process comprising extracting a raw extract from Emblica officinalis the improvement comprising conducting the extraction under conditions of time, temperature and atmosphere, to inhibit the formation of black specks and/or oligomeric and/or polymeric tannins and/or oxidation products thereof.

This invention relates to methods of eliminating undesired substances, including but not limited to oligomeric/polymeric components from compositions obtained from the fruit of Emblica officinalis plant also known as Phyllanthus Emblica and the resulting enriched compositions. This plant is generally found in India, China, Pakistan, Nepal and other countries. Accordingly, this invention is directed to extracts of Emblica officinalis from any geographical location.

Compositions obtained from an extract of the fruit of the Emblica officinalis plant have been described in the prior art, for example, in the above cross-referenced allowed application Ser. No. 10/120,156, the references referred to therein, as well as in U.S. Pat. No. 6,235,721 issued May 22, 2001 and U.S. Pat. No. 6,636,162 issued Mar. 26, 2002.

In U.S. Pat. No. 6,235,721, an anti-oxidant product referred to as “CAPROS” is isolated from the fruit of Emblica officinalis plant using a very dilute aqueous or alcoholic water salt solution, e.g. a 0.1 to 5% (w/w), preferably 1 to 2%, of a sodium chloride, potassium chloride, calcium chloride or magnesium chloride solution, which prevents degradation of the anti-oxidant compounds therein by enzymes present in the fruits of the Emblica officinalis plant. Alternatively, the anti-oxidant product is isolated using a buffer solution, e.g. 0.1 to 5% (w/w), preferably 1 to 2%, of sodium citrate/citric acid, sodium acetate/acetic acid, sodium phosphate/phosphoric acid, instead of aqueous or alcoholic water salt solution. It is further stated in this patent that the composition contains, by weight, Embilcanin-A and B (gallic/ellagic acid derivatives of 2-keto-glucono-δ-lactone) (35-55%), Punigluconin (2,3-di-O-galloyl-0,6-(S)-hexahydroxy-diphenoylgluconic acid) 4-15%), Pedunculagin (2,3,4,6-bis-(S)-hexahydroxydiphenoyl-D-glucose) (10-20%); Rutin (flavanol-3-)glycoside (5-15%); low to medium molecular weight gallo-ellagi tannoids (10-30%); gallic acid (0-5%) and ellagic acid (0-5%).

In the application U.S. Ser. No. 10/120,156, a standardized composition is described which is useful, for example, in skin lightening or skin whitening. This composition, hereinafter, termed “EMBLICA” is distinguished from “CAPROS”, by, for example, having less than 1% by weight of total flavonoids and even lower contents of RUTIN. Whereas, light colored EMBLICA consists essentially of the desired components for the purposes of skin lightening or skin whitening, it has been observed that black specks in the commercial product diminish the esthetic appearance of the final formulations. Other commercially available products based on extracts of Emblica officinalis are even darker in color due, on information and belief, to the presence of a larger number of black specks and water-insoluble oligomeric/polymeric materials.

Accordingly, one aspect of this invention is to provide at least one process for the removal of black specks in all types of extracts of Emblica officinalis so that the resulting composition is macroscopically (visually) devoid of such specks.

Another aspect of this invention is to provide a material substantially devoid of water-insoluble oligomeric/polymeric components.

We have discovered that the black specks are substantially, if not completely water-insoluble as measured at room temperature, (20-25° C.). A chemical analysis of these specks reveals that they comprise oligomeric/polymeric tannoids having no aromatic hydrogen.

We have also determined that the black specks have a particle size of on the order of about 20μ down to 1 micron Thus, it has been observed that some black specks pass through a 5 micron filter but hardly any pass through a one micron filter.

Without being bound by an explanation of the cause of the black specks, it is believed that the black specks are oxidation products, likely of phenolic hydroxy groups and/or oligomeric or polymeric tannins especially those having a molecular weight of above on the order of 3000.

We have discovered that such black specks and also oligomeric and polymeric tannins are substantially, if not completely water-insoluble, and that they are biologically inactive materials.

Thus, another aspect of this invention is to provide at least two processes which will remove the water-insoluble oligomeric and polymeric tannins, especially such tannins having a molecular weight of over 1000. preferably over 2000 and particularly over 3000 (hereinafter referred to as polymeric tannins). By water-insoluble it meant that a 1% by weight concentration of polymeric tannin in water does not exhibit a solubility of more that 10% by weight of the total tannin at 22° C.

Still another aspect is to provide substantially water-soluble (over 95% by weight) extracts of Phyllanthus Emblica comprising, for example, less than 5% by weight of polymeric tannins, with substantially no black specks and at high levels, e.g. over 70% by weight of bio-active, low molecular-weight hydrolysable tannins having molecular weights below 1,000. The resultant extracts can be used for all applications previously described in the prior art: e.g. in cosmetic formulations, for example, skin lightening or even-toning, anti-aging and sunscreens, as well as in nutritional supplements and any new applications developed in the future.

A powdered composition of Emblica officinalis wherein said composition is macroscopically substantially to completely devoid of black specks is a further embodiment of this invention.

A powdered composition of Emblica officinalis, wherein such composition contains at least 70% by weight of bio-active low molecular weight hydrolysable tannins is a further embodiment of this invention.

A powdered composition of Emblica officinalis, wherein the composition contains less than 5% by weight of oligomeric and polymeric tannins having a molecular weight of above 1000, preferably less than 5% by weight of oligomeric and polymeric tannins having a molecular weight of above 2000, and especially preferred less than 5% by weight of oligomeric and polymeric tannins having a molecular weight of above 3000 is a further embodiment of this invention.

The powdered compositions of Emblica officinalis can preferably comprise one or more water soluble diluents, preferably selected from the group comprising lactose, mannitol, dextrates, maltodextrin, dextrin, dextrose, and sucrose. The diluents are preferably present in an amount of 10 to 60% by weight.

Upon further study of the specification and dependent claims, further aspects and advantages of the inventions will become apparent.

To attain the objectives of the invention, there is provided at least one process which comprises preventing the formation of black specks and/or precursors thereof and/or polymeric tannins. Also provided is at least one process for separating the black specks and/or precursors thereof and/or the polymeric tannins from the remainder of the components of extracts of Emblica officinalis.

In general, the invention process comprises the following steps:

1) Providing an extract of Emblica officinalis either resulting from the original extract from the plant, or from a suspension of a powdered composition obtained after the extract is processed, e.g. after a drying step.

2) If necessary, physically separating the black specks and/or precursors thereof and/or polymeric tannins from the water-soluble components, for example by filtration with the use of a filter aid.

3) If desired, concentrating the resultant aqueous solution of the enriched composition of Emblica officinalis, for example to a dry powder.

With respect to step (1), if it is to be subjected to step (2), it is preferred to mix the raw extract or powdered extract with an aqueous solution preferably water. (By aqueous solution is meant water or mixture of water and a miscible solvent.) It is further preferred that the suspension contain about 5-30% more preferably about 18-22% by weight of total solids (including both dissolved and non-dissolved solids), and more preferably about 18-22%. When the extract is obtained from the fruit, the extraction is preferably conducted, under conditions so as to substantially prevent formation of polymeric tannins, e.g. low temperature (about 20° C. to 60° C.) and/or preferably under a substantially non-oxidizing atmosphere, e.g., the pressing apparatus is continuously flushed with nitrogen, and/or the addition of an autooxidation inhibitor, e.g. a saline solution. Likewise, the drying step is preferably conducted under conditions of temperature, time and atmosphere so as to mitigate the formation of black specks and/or polymeric tannins, examples of such conditions including but limited to drying at low temperature (freeze drying), short residence times in the spray drier, for example up to about 1 minute) and drying under vacuum at temperatures below 50° C.

If step (1) is nevertheless conducted under such conditions as to form black specks and/or precursors thereof, and/or polymeric tannins, it is necessary to conduct step (2).

As for step (2), the preferred separation method will take into account the physical and/or chemical properties of the black specks and/or precursors thereof. For example, as indicated above, in “EMBLICA”, the black specks have a particle size of approximately, of about 20μ or less.

Ideally, it would be preferred to provide a method of separation which retains the bio-active components of EMBLICA by removing only the undesired components. Whereas there are a variety of separation procedures that can be employed, e.g. any one of a number of well-known filtration or centrifugation processes or combinations thereof, it is also contemplated that still other separation processes can be employed such as, for example, sedimentation, flotation and elutriation. A filter aid, e.g. diatomite filter aids, cross-linked polyvinyl pyrrolidone as well as silica and silicate sorbents can also be used to remove the oligomeric/polymeric materials. Some of the suppliers of these filter aids are Advanced Minerals (Celpure 25, 65 & 100, AW Cellite NF, MP Harborlite), International Specialty Products (Plasdone XL), United Perlite Corporation (Ultralite Perlite 505, 606C, 606F, 808, 909C, 909F). Likewise, extraction of the black specks or precursors thereof with a substantially water-miscible solvent, e.g. (ethanol, methanol, isopropanol or mixture of solvents) is also contemplated. For further details of separation systems, reference is made to descriptions in the patent and chemical engineering literature, for example, section 19 (liquid-solid systems) in Perry's Chemical Engineer's Handbook, 6th edition, editors Perry, Green and Maloney, 1984, McGraw-Hill Book Company.

With respect to step (3), a concentrated composition of water-soluble EMBLICA components can be produced by any number of conventional chemical engineering drying techniques. e.g. those described in Section 20 of Perry's Chemical Engineer's Handbook, 6th edition, and including but not limited to tray dryers, rotary dryers, agitated dryers, gravity dryers, vibrating-conveyor dryers, pneumatic conveyor dryers, Glatt dryers, freeze dryers and spray dryers. It is contemplated that prior to the drying step that the aqueous solution of the desired Emblica offinalis components can optionally be subjected to evaporation under sufficiently low temperatures so as to not to deleteriously affect the components. In view of the nature of the components, it is contemplated in order to forestall decomposition during drying that drying under vacuum, e.g.—freeze drying, will be preferred over a high temperature spray drying technique.

Without intending to be bound by the chemical structure, the water-insoluble oligomeric/polymeric components of Phyllanthus emblica extract appear to be based on the following general structure of monomeric units: 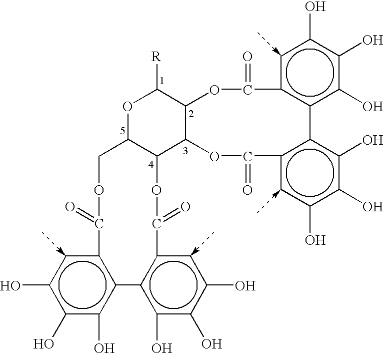
wherein R represents OH or ═O; and C-2/C-3 can have an unsaturation.

The arrow heads indicate the points of substitution meaning a fully aromatic-substituted product. The substituted moieties comprise other monomeric units which can be attached via a C—C bond and/or a C—O bond.

As for the evidence of the above depicted structure, the 300 MHz ¹H-NMR spectrum of the acetylated product, in CDCl₃ showed complete absence of Aromatic H signals. It is important to note that these oligomeric/polymeric tannins may create adverse health problems as they can combine irreversibly with some proteins. Hence, their presence is to be avoided.

One process to avoid the formation of oligomeric/polymeric tannins comprises the introduction of a small amount of salt solution, preferably sodium or potassium chloride, during the processing of the fruit juice. This salt solution inhibits the facile autooxidation of the small gallo-ellagi tannins into oligomeric/polymeric tannins. In addition to sodium or potassium chloride, it is contemplated that the addition of any non-reactive, soluble, ionizable compounds will increase the ionic strength of the reaction solution and will therefore inhibit oligomerization/polymerization.

By substituting the enriched compositions of Emblica officinalis produced by the present invention for the non-enriched Emblica extracts, substantial advantages are obtained. Examples of such compositions include but are not limited to skin and personal care compositions, e.g. sunscreens, as well as pharmaceutical and nutritional compositions.

Without further elaboration, it is believed that one skilled in the art, can, using the preceding description, utilize the present invention to its fullest extent. The following embodiments are, therefore, to be construed as merely illustrative and not limitative of the remainder of the disclosure in any way whatsoever. (These embodiments have not been necessarily actually conducted or prepared.)

### EXAMPLES

### Example 1

A 20% by weight of an aqueous dispersion of EMBLICA powder was prepared by mixing the EMBLICA in water in a stainless-steel container with a hand-held agitator for about 15 minutes in order to obtain an uniform dispersion.

The properties of the EMBLICA powder, were as follows:

Low molecular weigh tannins −77.8% by HPLC

Water insoluble material −12.2%

Pale yellow powder

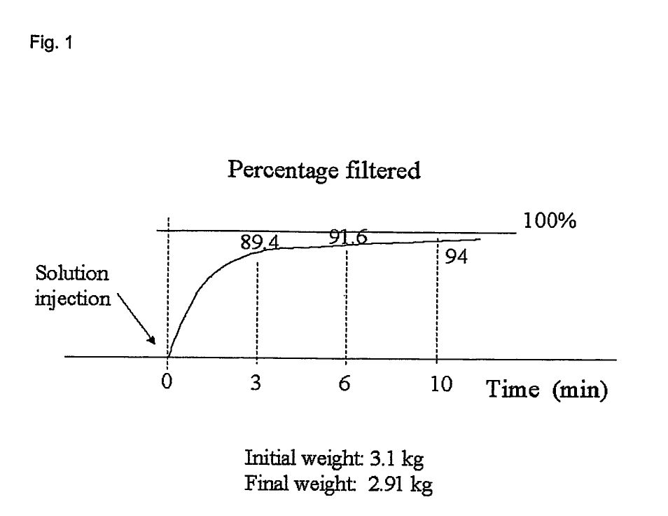

The resultant dispersion was then subjected to centrifugal filtration using a centrifuge (Heinkel HF 300, bowl diameter 300 mm, filter area 0.1 m²). 3L of a 10% solution of EMBLICA were filtered at a centrifuge speed of 1500 rpm. The filtration was complete within 10 min and yielded the curve of weight filtration diplayed in FIG. 1. The filter cloth porosity was 5 μm.

Filtered material was dried to a powder using a spray drier.

### Example 2

Using the same centrifuge employed in Example 1, two tests were performed at a 33% by weight concentration of EMBLICA in purified water but with different centrifugation speeds, i.e. different g forces applied to the product. Two first tests of 3 L each were filtered at 1500 rpm (˜375 g) and the liquid recovered. In a second step, 8 L were filtered in several parts at 3000 rpm (˜1500 g) to determine if further liquid extraction can be achieved. A filter of 1 μm porosity (model 3 54 FC) was chosen since it gave a reasonable liquid cross-flow. Also, this is the same filter used in previous filtration test with a 20% solution which gave good results. The EMBLICA used in these tests have same characteristics as described in Example 1.

Test 1

3 L of a 33% solution of EMBLICA were filtered at a centrifuge speed of 1500 rpm. The filtration was slower than at 20% but almost complete after 15 min. The resulting filtrate solution constituting about ⅔ by weight of the original solution was opaque and about 70% initial material was recovered. In order to increase the recovery, a second test was made a higher centrifugation speed. No black particles were visually (macroscopically) observed in the filtrate but many were observed on the residue on the filter.

Test 2

33% EMBLICA solution was filtered by using the same filter but a higher centrifugation speed of 3000 rpm. Filtration was only slightly improved despite a 4 times higher g force. Out of 12 kgs of initial material, only 8.3 kgs were obtained. No black particles were observed in the filtrate solution. Accordingly, filtration tests with a 33% w/w solution of EMBLICA show satisfactory elimination of black particles, similar to previous tests with 10 and 20% solutions. However, 33% weight concentration appears too high for maximal product throughput. Filtration at 18-22% is therefore preferred.

### Example 3

The solutions of Example 2 obtained by filtration at 1500 and 3000 rpm were spray dried separately. Conditions were an inlet temperature of 345±5° F., an outlet temperature of 230±5° F. and a feed rate of 100 ml/min. The spray drier was a 30 inch Bowen Lab unit.

The laboratory results were as follows:

Processed first:

3000 rpm solution INPUT: 8.2 kgs OUTPUT: 1.395 kgs (+1.2 kgs) followed by

1500 rpm solution INPUT: 3.7 kgs OUTPUT: 1.06 kgs (+0.37 kgs)

The OUTPUT weights correspond to the direct product obtained as well as the weight of sticking product brushed off the vessel's walls. The latter product caused by hot steel walls of the vessel shows a clearly darker color (orangish-brownish) than the direct dried product (off-white to light beige). To overcome such sticking it is contemplated that production vessels will include an additional insulation of the walls which will reduce, if not eliminate, this effect. No significant loss of material occurs during the spray drying process. The resulting product powder is quite dry, fluffy and slightly whiter than the original.

The highest product loss occurred during the centrifuge filtration step due to the high initial concentration in the test. A much higher filtration throughput can be obtained by using a 20%/w. solution.

The following table provides a chromatographic analysis of 2 lots.

### Emblica™ (Centrifuge-Spray Dried Sample): Calculation of Actives from HPLC Data

$$\% small tannoids = \frac{Σ areas of active peaks}{Total area of the chromatogram}$$

Lot No. F15

Areas for the actives: Emblicanin A+Emblicanin B+Punigluconin+Pedunculagin=Areas of peaks 1, 3, 6, 8=894.95+513.28+261.87+891.97=2,562.07 Total area per HPLC: 2,929.93

% of actives=2,562.07/2,929.93=87.45%

% of Emblicanin A=894.95/2,929.93=30.55%

% of Emblicanin B=513.28/2,929.93=17.52%

% of Punigluconon=261.87/2,929.93=8.95%

% of Pedunculagin=891.97/2,929.93×83.80=30.44%

Lot No. F30

Areas for the actives: Emblicanin A+Emblicanan B+Punigluconin+Pedunculagin+Areas of peaks 1, 3, 6, 8=904.51+502.66+251.66+889.70=2,548.53

Total area per HPLC: 2,995.03

% of actives=2,548.53/2,995.03=85.10%

% of Emblicanin A=904.51/2,995.03=30.20%

% of Emblicanin B=502.66/2,995.03=16.79%

% of Punigluconon=251.66/2,995.03=8.40%

% of Pedunculagin=889.70/2,995.03=29.70%

### Example 4

A 20% by weight of an aqueous dispersion of EMBLICA powder (100 Kg) was prepared by mixing the EMBLICA in water in a stainless-steel vessel filled with a mechanical agitator for about 1 hr in order to obtain an uniform dispersion. Then about 5 Kg of a diatomite filter aid (Celpure 1,000) was blended well to bind oligomeric/polymeric tannins. The slurry was mixed for approximately 30 min at room temperature. The residue was removed by centrifugation (i.e., in a Beckman™ J6B swinging one liter bucket rotor at 3000 rpm for 5 min), or by pressure filtering (i.e., through a coarse cellulose Cuno™. CPX-01A depth filter pad, with a pressure of 5 psi, 35 kPa). The filtered aqueous solution was then dried either by using a freeze drier or a spray drier.

### Example 5

A 15% by weight of an aqueous dispersion of EMBLICA powder (10 Kg) was prepared by mixing the EMBLICA in water in a stainless-steel vessel filled with a mechanical agitator for about 1 hr in order to obtain a uniform dispersion. Slight heating to about 30 to 40 C can expedite the process of dispersion. Then about 0.5 to 1 Kg of a diatomite filter aid (Celpure 1,000) was blended well to bind oligomeric/polymeric tannins. The slurry was mixed for approximately 30 min at room temperature and was allowed to stay for about 5 to 10 hrs. Almost clear liquid on the top was siphoned-off and then passed through a coarse filtration (cheese cloth) to remove any undesirable insoluble particulates. The aqueous solution was then dried either by using a freeze drier or a vacuum drier (at about 55-60 C).

### Example 6

The Emblica antioxidant fraction is obtained directly from the fruits by following a three-step process: (1) Extraction: Emblica officinalis fruits were extracted with water or by squeezing the fruit flesh. (2) Removal of water-insoluble material: The fresh water-extract or the juice was then subjected to centrifugation and the supernatant is siphoned-off. Alternately, the water extract or the juice was admixed with a filter-aid and then filtered to remove the water-insoluble material. (3) Drying: The water-soluble fraction was then dried under vacuum or freeze dried.

HPLC analysis showed that the powder of Emblica antioxidant fraction contains 74.3% low molecular-weight hydrolysable tannins, the key bioactive components of the invention.

### Example 7

### Skin Care Lotion

| | TRADE | |
|---|---|---|
| INCI NAME | NAME/MANUFACTURER | % w/w |
| Phase A | | |
| Water (demineralized) | | 65.97 |
| Disodium EDTA | | 0.10 |
| Propylene Glycol | | 2.00 |
| Sorbitol | Sorbo (70% soln.)/Uniqema | 2.00 |
| Sodium Lauryl Sulfate | Stepanol ME-Dry/Stepan | 0.15 |
| Phase B | | |
| Glyceryl stearate | Tegin M/Goldschmidt | 5.00 |
| Stearic acid | Emersol 132/Cognis | 1.00 |
| Persea Gratissima (Avocado) | Crodarom Avocadin/Croda | 15.00 |
| oil Unsaponifiables | | |
| Beeswax | White Bleached NF Beeswax | 1.50 |
| | Prills/Ross | |
| Phase C | | |
| Water (demineralized) | | 5.00 |
| Phyllanthus emblica fruit extract | Present Invention* | 1.00 |
| Phase D | | |
| Triethanolamine | TEA 99%/Union Carbide | 0.28 |
| Phase E | | |
| Propylene glycol, DMDM | Paragon/Mc Intyre | 1.00 |
| Hydantoin, Methylparaben | | |
| Total | | 100.00 |

Procedure: Combine A and heat to 70-75° C. Combine B and heat to 70-75° C. Add B to A while stirring. Add phase C at 30° C. Adjust pH to 5.0-6.0 with phase D. Add phase E. Mix until uniform.
*By “Present Invention” is meant the enriched EMBLICA having a decreased concentration of black specks and oligomer/polymers.

### Example 8

### Skin Lightening Lotion

| | TRADE | |
|---|---|---|
| INCI NAME | NAME/MANUFACTURER | % w/w |
| Phase A-1 | | |
| Water (demineralized) | | 56.18 |
| Disodium EDTA | | 0.05 |
| Propylene Glycol | | 5.00 |
| Phase A-2 | | |
| Xantham Gum | Vanzan NF/Vanderbilt | 0.25 |
| Magnesium aluminum stearate | Veegum Ultra | 0.40 |
| | granules/Vanderbilt | |
| Phase B | | |
| Cetearyl alcohol and cetearyl | Montanov 68/Seppic | 7.00 |
| glucoside | | |
| Apricot kernel oil | Lipovol P/Lipo | 10.00 |
| Octyl stearate | Cetiol 868/Cognis | 3.00 |
| Dimethicone | Dow Corning 200 Fluid | 6.00 |
| | 10 cst/Dow Corning | |
| Phase C | | |
| Water (demineralized) | | 10.00 |
| Phyllanthus emblica fruit extract | Present Invention | 1.00 |
| Phase D | | |
| Triethanolamine | TEA 99%/Union Carbide | 0.12 |
| Phase E | | |
| Phenoxyethanol, | Liquapar PE/Sutton | 1.00 |
| Isopropylparaben, | | |
| Isobutylparaben, Butylparaben | | |
| Total | | 100.00 |

Procedure: Disperse A-2 in A-1 and heat to 70-75° C. Combine B and heat to 70-75° C. Add B to A while stirring. Homogenize until mixture cools to 60° C. At 30° C. add phase C. Adjust pH with TEA to 4.0-5.0. Add phase E. Mix until uniform.

### Example 9

### Skin Lightening Lotion

| | TRADE | |
|---|---|---|
| INCI NAME | NAME/MANUFACTURER | % w/w |
| Phase A-1 | | |
| Water (demineralized) | | 55.05 |
| Disodium EDTA | | 0.05 |
| Propylene Glycol | | 5.00 |
| Phase A-2 | | |
| Xantham Gum | Vanzan NF/Vanderbilt | 0.25 |
| Magnesium aluminum stearate | Veegum Ultra | 0.40 |
| | granules/Vanderbilt | |
| Phase B | | |
| Cetearyl alcohol and cetearyl | Montanov 68/Seppic | 7.00 |
| glucoside | | |
| Apricot kernel oil | Lipovol P/Lipo | 10.00 |
| Octyl stearate | Cetiol 868/Cognis | 3.00 |
| Dimethicone | Dow Corning 200 Fluid | 6.00 |
| | 10 cst/Dow Corning | |
| Phase C | | |
| Water (demineralized) | | 10.00 |
| Phyllanthus emblica fruit extract | Present Invention | 2.00 |
| Phase D | | |
| Triethanolamine | TEA 99%/Union Carbide | 0.25 |
| Phase E | | |
| Phenoxyethanol, | Liquapar PE/Sutton | 1.00 |
| Isopropylparaben, | | |
| Isobutylparaben, Butylparaben | | |
| Total | | 100.00 |

Procedure: Disperse A-2 in A-1 and heat to 70-75° C. Combine B and heat to 70-75° C. Add B to A while stirring. Homogenize until mixture cools to 60° C. At 30° C. add phase C. Adjust pH with TEA to 4.0-5.0. Add phase E. Mix until uniform.

### Example 10

### Age-Defying Lotion

| | TRADE | |
|---|---|---|
| INCI NAME | NAME/MANUFACTURER | % w/w |
| Phase A-1 | | |
| Water (demineralized) | | 59.15 |
| Disodium EDTA | | 0.05 |
| Propylene Glycol | | 5.00 |
| Phase A-2 | | |
| Xantham Gum | Vanzan NF/Vanderbilt | 0.20 |
| Phase B | | |
| PEG-6 stearate, ceteth-20, | Tefose 2561/Gattefosse | 10.00 |
| glyceryl stearate, steareth-20, | | |
| stearic acid | | |
| Stearic Acid | Emersol 132/Cognis | 1.00 |
| Hydrogenated castor oil | Cutina HR/Cognis | 1.00 |
| Octyldodecyl myristate | M.O.D./Gattefosse | 8.00 |
| Dimethicone | Dow Corning 200, 50 | 4.00 |
| | cst/Dow Corning | |
| Phenyltrimethicone | Dow Corning 556 Wax/Dow | 2.00 |
| | Coning | |
| Sweet Almond oil | Cropure Almond/Croda | 3.00 |
| Phase C | | |
| Water (demineralized) | | 5.00 |
| Phyllanthus emblica fruit extract | Present Invention | 0.50 |
| Phase D | | |
| Triethanolamine | TEA 99%/Union Carbide | 0.10 |
| Phase E | | |
| Phenoxyethanol, | Liquapar PE/Sutton | 1.00 |
| Isopropylparaben, | | |
| Isobutylparaben, Butylparaben | | |
| Total | | 100.00 |

Procedure: Disperse A-2 in A-1 and heat to 70-75° C. Combine B and heat to 70-75° C. Add B to A while stirring. Homogenize until mixture cools to 60° C. At 30° C. add phase C. Adjust pH with TEA to 5.0-6.0. Add phase E. Mix until uniform.

### Example 11

### Sunscreen Lotion

| | TRADE | |
|---|---|---|
| INCI NAME | NAME/MANUFACTURER | % w/w |
| Phase A | | |
| Butylmethoxydibenzoylmethane | Eusolex 9020/Rona | 1.00 |
| Glyceryl Stearate, Ceteareth-15 | Tegocare 215, Pellets/Degussa | 3.00 |
| Decyl oleate | Cetiol V/Cognis | 5.00 |
| Isopropyl palmitate | Isopropyl palmitate | 5.00 |
| Dimethicone | Mlrasil DM 350 | 0.50 |
| Stearyl alcohol | Lanette 18 | 2.00 |
| Carbomer | Carbopol ETD 2050 | 0.10 |
| Phase B | | |
| Glycerin | Glycerol (about 87%) | 3.00 |
| Ectoin | RonaCare Ectoin/Rona | 0.50 |
| Phenoxyethanol, | Liquapar PE/Sutton | 1.00 |
| Isopropylparaben, | | |
| Isobutylparaben, Butylparaben | | |
| Aqua (water), Ethyhexyl | | 15.00 |
| metoxycinnamte, Silica, PVP, | | |
| Chlorphenesin, BHT | | |
| Water, demineralized | Aqua (water) | qs |
| Phase C | | |
| Phyllanthus emblica fruit extract | Present Invention | 0.50 |
| Phase D | | |
| Sodium hydroxide | Sodium hydroxide, 10% | 0.45 |
| | solution | |
| Phase E | | |
| Perfume | Fragrance delicat/Drom | 0.20 |
| Total | | 100.00 |

Procedure: Heat phases A and B separately to 80 C. stir phase A. Homogenize. At 30 C, add phase C. Adjust pH with sodium hydroxide to 5.5. Finally add phase E to the emulsion.

### Example 12

### Anhydrous Oil-Free Gel

| | TRADE | |
|---|---|---|
| INCI NAME | NAME/MANUFACTURER | % w/w |
| Phase A | | |
| Ozokerite | White Ozokerite SP-1020/Strahl & | 3.00 |
| | Pitsch | |
| Cyclomethicone | Dow Corning 345 Fluid/Dow | 25.00 |
| | Corning | |
| Cyclomethicone (and) | Gransil GCM/Grant Industries | 60.00 |
| Polysilicone-11 | | |
| Phase B | | |
| Bismuth Oxychloride | Biron ® LF-2000/Rona | 2.00 |
| Phase C | | |
| Cyclomethicone | Dow Corning 345 Fluid/Dow | 3.60 |
| | Corning | |
| Cyclomethicone (and) | Dow Corning 9040 Silicone | 5.40 |
| Dimethicone Crosspolymer | Elastomer Blend/Dow Corning | |
| Present Invention | | 1.00 |
| Total | | 100.00 |

Procedure: Blend ingredients in Phase A; heat with mixing until clear and uniform. Add bismuth oxychoride and disperse with mixing. Blend ingredients in Phase C separately; the mixture should be smooth and contain no lumps. Cool Phase A/B to 50-60° C. and add Phase C with mixing. When the mixture is uniform it may be packaged.

### Example 13

### Capsules & Tablets

Procedure: 1 is granulated with starch paste to make it a free flowing powder. Blend all the ingredients, except 4, for 25 min. in a blender. Screen in 4 and blend for an additional 5 min. Compress into tablets using 7/16 in standard concave tooling. Alternately, the blended material can be filled into appropriate capsules.

### Example-14

### Tablets and Capsules

| | Composition | Quantity per |
|---|---|---|
| Ingredient | (w/w, in %) | tablet (mg) |
| 1. Inventive Composition | 60.0 | 250.0 |
| 2. Avicel pH 101 | 20.0 | 84.0 |
| 3. Starch 1500 or | 17.5 | 75.5 |
| Maltodextrin | | |
| 4. Steric acid, N.F. (powder) | 2.0 | 8.5 |
| 5. Cab-O-Sil | 0.5 | 2.0 |

Note:
Emblica officinalis water soluble extract is granulated with starch paste or maltodextrin to make it a free-flowing powder.

Procedure: Blend all the ingredients, except 4, for 25 min. in a blender. Screen in 4 and blend for an additional 5 min. Compress into tablets using 7/16 in standard concave tooling. Alternately, the blended material can be filled into appropriate capsules.

### Example 15

### Chewable Tablets

| | Composition | Quantity per |
|---|---|---|
| Ingredient | (w/w, in %) | tablet (mg) |
| 1. Inventive Composition | 9.26 | 26.60 |
| 2. Sodium ascorbate, USP | 36.26 | 81.60 |
| 3. Avicel pH 101 | 19.12 | 38.50 |
| 4. Sodium saccharin, (powder), N.F. | 0.56 | 1.25 |
| 5. DiPac | 29.30 | 66.00 |
| 6. Stearic acid, N.F. | 2.50 | 5.60 |
| 7. Imitation orange Flavor | 1.0 | 2.25 |
| 8. FD & C Yellow #6 dye | 0.5 | 1.12 |
| 9. Cab-O-Sil | 0.5 | 1.12 |

Procedure: Blend all the ingredients, except 6, for 20 min in a blender. Screen in 6 and blend for an additional 5 min. Compress into tablets using 7/16-in standard concave tooling.

### Example 16

### Beverages

| Ingredient | Quantity per 500 ml |
|---|---|
| 1 Present Invention | 10 mg-2 gm |
| 2 Excipients: Carbonated Water, Food Starch-q.s, | |
| Modified, High Fructose Corn Syrup and/or Sucrose | |
| and/or Sugar, Sodium Benzoate, Caffeine, | |
| Glycerol Ester of Wood resin, Flavors, Colors | |

### Example 17

### Cereals

| Ingredient | Quantity per 1 Kg |
|---|---|
| 1. Extract of Invention | 500 mg-10 gm |
| 2. Excipients: Whole Grain Oats, Oat Bran, q.s, Sugar, | |
| Modified Corn Starch, Brown Sugar Syrup, Salt, | |
| Calcium Carbonate, Trisodium Phosphate, Wheat | |
| Flour, Vitamin E (Mixed tocopherols), Zinc & Iron | |
| Mineral nutrients), Niacinamide (A B Vitamins), | |
| Vitamin B6 (Pyridoxine HCl), Vitamin B2 | |
| Riboflavin), Vitamin B1 (Thiamin Mononitrate), | |
| Vitamin A (Palmitate), Vitamin A B (Folic acid), | |
| Vitamin B12, Vitamin D | |

### Example 18

### Maintenance Multivitamin Tablets and Capsules

| | | Composition | Quantity per |
|---|---|---|---|
| Ingredient | | (w/w, in %) | tablet (mg) |
| 1. | Vitamin A acetate | 5.5 | 11.0 |
| 2. | Thiamine mono-nitrate, USP | 0.8 | 1.65 |
| 3. | Riboflavin, USP | 1.1 | 2.10 |
| 4. | Pyridoxine HCl, USP | 1.0 | 2.10 |
| 5. | 1% Cyanocobalamine (in gelatin) | 1.0 | 2.10 |
| 6. | D-Calcium pantothenate, USP | 3.75 | 7.50 |
| 7. | Inventive Composition, free-flowing | 32.25 | 65.50 |
| 8. | Niacinamide | 11.0 | 22.00 |
| 9. | DiTab | 13.1 | 26.20 |
| 10. | Microcrystalline cellulose, N.F. | 25.0 | 50.00 |
| 11. | Talc, USP | 3.0 | 6.00 |
| 12. | Stearic acid, (powder), N.F. | 1.5 | 3.00 |
| 13. | Magnesium stearate, (powder), N.F. | 1.0 | 2.00 |

Procedure: Blend all ingredients for 20 min in a suitable blender. Screen in 12 and blend for an additional 5 min. Compress at a tablet weight of 200 mg using ⅜-in standard concave tooling. Alternately, blended material is filled into a capsule containing 200 mg of multi-vitamins. These tablets or capsules can be used as nutritional supplements.

Notwithstanding the details of the preceding embodiments, it is to be understood there are several broad concepts in the present invention.

The first broad concept relates to the treatment of a raw extract from Emblica officinalis. Once it is known that it is important to adjust the time, and/or temperature, and/or atmosphere and/or chemistry of the conditions of the extraction as to inhibit the formation of polymeric tannins and/or black specks, a chemical engineer or the like would be able to adjust such variables so as to inhibit the formation of the undesired components. This would require measuring the extent of the undesired components without adjustment of the variables and then adjusting the variables so as to provide an improved process. For example, lower temperatures and shorter residence times should result in a lower degree of oligomerization or polymerization. Likewise, the less oxygen in the atmosphere, the less likelihood of oxidation to form undesired impurities. Consequently, by adjusting at least one of the variables, it is possible that only one variable need be adjusted in order to obtain the desired inhibition, for example, temperature. Nevertheless, it is also contemplated that two or more variables may also be adjusted so as to arrive at the optimum conditions.

Another basic concept of the invention relates to concentrating the extract, e.g. in order to form a powder. Again, the temperature, time and atmosphere in which the concentrating is conducted will have an effect on the degree of impurities in the resultant dried composition. Consequently, a chemical engineer or the like will be able to adjust at least one of the variables in order to obtain a product which is substantially to completely devoid of black particles when viewed visually (macroscopically), preferably at least 95%, more preferably at least 99%). By “substantially devoid” is meant that the black particles are decreased in number compared to the number of black particles which would be present in the absence of the adjustment of the variables. Preferably, the composition should be completely devoid of black specks) but it is contemplated that it would be sufficient for esthetic purposes for the composition to contain not more than 100, preferably below 10 black specks per 500 grams of composition).

Another concept of the invention relates to the reduction of potentially biologically adverse components in the extract. This is accomplished, for example, by removing at least a portion of polymeric tannins having a molecular weight of above 1,000, and especially above 3000.

Thus, taking into consideration the various concepts and aspects of the invention, the preceding examples can be repeated with substantially similar success by substituting generically or specifically described steps and/or operating conditions for those set forth in the examples.

The entire disclosure of all applications, patents, and publications cited above, including those references set forth in said applications, patents and publications are hereby incorporated by reference.

From the foregoing description, one skilled in the art can easily ascertain the essential characteristics of this invention and without departing from the spirit and scope thereof, can make various changes and modifications of the invention to adapt it to various usages and conditions.

Short Explanation of the Figures

FIG. 1: Curve of weight filtration obtained according to Example 1

1. Extraction process comprising extracting a raw extract from Emblica officinalis wherein the extraction is conducted under conditions of time, temperature and atmosphere, to inhibit the formation of black specks and/or oligomeric and/or polymeric tannins and/or oxidation products thereof.

2. A process according to claim 1, comprising inhibiting the formation of said oilgomeric and/or polymeric tannins having a molecular weight above 1000.

3. Process of producing a powdered extract of Emblica officinalis, comprising the step of drying an aqueous solution of the extract, wherein the drying is conducted under conditions of time, temperature and atmosphere so as to inhibit the formation of oligomeric and/or polymeric tannins and/or oxidation products thereof and/or black specks.

4. A process according to claim 3 comprising inhibiting the formation of said oligomeric and/or polymeric tannins having a molecular weight above 1000.

5. A process comprising enriching an extract of Emblica officinalis, comprising the steps of:
A) providing an aqueous suspension of Emblica officinalis, said aqueous suspension containing dissolved components of Emblica officinalis and water-insoluble components comprising black specks and/or oligomeric and polymeric tannins; and
B) separating the insoluble components from said dissolved components to obtain an enriched aqueous extract of Emblica officinalis.

6. A process according to claim 5, further comprising a preceding step of dispersing a powdered extract of Emblica officinalis in an aqueous solution to form said aqueous suspension.

7. A process according to claim 5, wherein said separating comprises subjecting said suspension to centrifugation or pressure filtration.

8. A process according to claim 5, wherein said separating comprises adding a filter-aid to said aqueous suspension and filtering out the insoluble components of the suspension so as to obtain an enriched aqueous filtrate of Emblica officinalis.

9. A process according to claim 5, wherein said aqueous suspension has a concentration of 5-30% by weight, preferably 18-22% by weight of total solids of Emblica officinalis.

10. A process according to claim 5, comprising subjecting said aqueous suspension to sufficient centrifugation to obtain an enriched extract of Emblica officinalis macroscopically substantially to completely devoid of black specks.

11. A process according to claim 5, further comprising drying the separated enriched aqueous extract of Emblica officinalis.

12. A process according to claim 5, wherein said drying step comprises spray drying or freeze drying.

13. A process according to claim 5, comprising drying said solution of Emblica officinalis under conditions of time, temperature and atmosphere so as to inhibit the formation of oligomeric and/or polymeric tannins having a molecular weight of over 1000.

14. An extract of Emblica officinalis produced by the process of claim 1

15. Powdered composition of Emblica officinalis, the improvement wherein said composition is macroscopically substantially to completely devoid of black specks.

16. Powdered composition of Emblica officinalis, wherein such composition contains at least 70% by weight of bio-active low molecular weight hydrolysable tannins.

17. Powdered composition of Emblica officinalis, wherein the composition contains less than 5% by weight of oligomeric and polymeric tannins having a molecular weight of above 1000, preferably less than 5% by weight of oligomeric and polymeric tannins having a molecular weight of above 2000, and especially preferred less than 5% by weight of oligomeric and polymeric tannins having a molecular weight of above 3000.

18. Powdered composition of Emblica officinalis according to claim 15, characterized in that the composition comprises one or more water soluble diluents, preferably selected from the group comprising lactose, mannitol, dextrates, maltodextrin, dextrin, dextrose, and sucrose, wherein the diluents are preferably present in an amount of 10 to 60% by weight.

19. A skin or hair or personal care composition formed from ingredients comprising a powdered composition according to claim 15.

20. A skin or personal care composition according to claim 19 in the form of a lotion, creme, stick, spry or gel.

21. A skin or personal care composition according to claim 19 which contains about 0.05 to 5% of said tannins.

22. A pharmaceutical or nutritional composition formed from ingredients comprising a powdered composition according to claim 15 in the form of a tablet, capsules, elixir, syrup, or drinks.

23. A pharmaceutical or nutritional composition according to claim 22 formed from about 0.05 to 20% of said powdered composition.
